(19) United States
(12) Patent Application Publication
(10) Pub. No.: US 2026/0097086 A1
(43) Pub. Date: Apr. 9, 2026

(54) COMPOSITIONS AND USES THEREOF

(71) Applicant: SKINBIOTHERAPEUTICS PLC, Newcastle upon Tyne (GB)

(72) Inventor: Catherine O'NEILL, Cheshire (GB)

(21) Appl. No.: 19/113,160

(22) PCT Filed: Sep. 20, 2023
(86) PCT No.: PCT/GB2023/052431

§ 371 Date: Mar. 19, 2025

(30) Foreign Application Priority Data
Sep. 20, 2022 (GB) 2213726.9

(51) Int. Cl. A61K 35/74; A61K 8/99; A61P 17/00; A61Q 19/00

(52) U.S. Cl. CPC A61K 35/74; A61K 8/99; A61P 17/00; A61Q 19/004

(57) ABSTRACT

The present invention relates to a composition comprising a heterotrophic skin bacterium and/or heterotrophic skin bacterial extract for use in the modulation of cytokine expression in a skin cell. The composition is particularly suited for use in the prevention and/or treatment of skin damage caused by UV light and use in treating skin conditions such as polymorphous light eruption (PLE).

### FIELD OF THE INVENTION

The present invention relates to compositions comprising a heterotrophic skin bacterium and/or heterotrophic skin bacterial extracts for use in the modulation of cytokine expression in a skin cell. In particular, the heterotrophic skin bacterium is Micrococcus luteus (M. luteus) and/or the heterotrophic skin bacterial extract is derived from M. luteus. The compositions are well suited for use in the mediation of cytokine expression in the skin. In particular, the compositions are suited for use in the prevention and/or treatment of skin conditions linked to cytokine expression such as those caused by skin exposure to UV light.

### BACKGROUND TO THE INVENTION

The mechanisms used by the bacteria to exert positive effects on humans are varied and include inhibition of pathogens, modulation of the immune response and enhancement of epithelial barrier function. However, in general, the nature of the underlying probiosis is poorly characterised and there is comparatively little information available regarding the molecular mechanisms mediating the observed effects of probiotics.

The topical application of probiotics to the skin has been investigated in a limited number of studies. In general, the mechanisms underlying these effects are not well understood.

Current theories indicate that immune regulation involves homeostasis between T-helper 1 (Th1) and T-helper 2 (Th2) activity with Th1-and Th2-helper cells directing different immune response pathways. Overactivation of either pattern can cause disease, and either pathway can down-regulate the other. Th1 and Th2 pathways are generally associated with secretion of different cytokines.

Exposure to ultraviolet radiation (UVR) suppresses the immune response. Data from a number of laboratories have indicated that one consequence of UVR exposure is suppressed T helper type 1 (Th1) cell function with normal Th2 cell activation, resulting in a shift to a Th2-like phenotype. Furthermore, there are a number of conditions that are exacerbated or induced by exposure to UVR. These are collectively known as the photodermatoses and includes conditions such as urticaria, photoaggravated eczema and polymorphous light eruption (PLE or PMLE).

Polymorphous Light Eruption (PLE or PMLE) is a skin “complaint” caused by sunlight. The name derives from polymorphic eruption, which means a rash that has many forms. PLE is thought to be caused by an immune reaction to a compound in the skin which is altered by exposure to ultraviolet radiation. PLE occurs with typical sun exposure of 2 to 3 days but can also happen at lesser exposures (like direct exposure to incident rays through windows or 15 to 20 minutes in direct sun).

A delayed-onset, spotty, itchy eruption appears on the skin which may take 5 to 10 days to clear. The rash usually consists of small red spots or blisters and can appear on any part of the body that has been exposed to sunshine, although commonly the face and the backs of the hands will be spared. It tends to heal without scarring. PLE is different to prickly heat which affects the trunk due to warm weather rather than exposure to sunlight.

PLE is observed in 20-30% of the northern European and U.S. population (places where frequent sun exposure is uncommon) and 10 to 20% of the population elsewhere, with females being affected more than males. It generally affects patients by the age of 30 with a median age range between 20 and 40. The problem is more common in countries with temperate climates like those of Europe and America, generally from the onset of spring through summer. PLE is most likely related to an immune system reaction in the skin of certain people, more commonly with those having a fair skin. Evidence of PLE has also been found in northern regions of China, and Australia. Current data suggests that the development of photodermatoses like PLE may be related to excess expression of TH1 and suppression of TH2 responses. There is no proven therapy to help alleviate skin disorders like polymorphic light eruption (PLE) to date although calamine lotions, topical steroid creams containing corticosteroid, hydrocortisone and hydroxychloroquine along with some regular itching medicines are currently used with limited efficacy.

There is currently no effective treatment for PLE with the majority of therapies and compositions designed to alleviate the symptoms rather than to treat or prevent the condition itself. For at least the reasons set out above there is a need to develop compositions, therapies and methods to prevent and/or treat and/or ameliorate PLE and skin conditions caused by skin exposure to UV.

### SUMMARY OF THE INVENTION

The invention is defined in the appended claims and also includes the combination of the aspects and preferred features hereinafter described except where such a combination is clearly impermissible or expressly avoided.

In accordance with an aspect of the present invention, there is provided a composition comprising a heterotrophic skin bacterium and/or a heterotrophic skin bacterial extract for use in the modulation of cytokine expression in a skin cell. As will be appreciated, in embodiments in which the composition comprises a heterotrophic skin bacterium, the composition may contain whole/intact bacteria. Alternatively, in embodiments the heterotrophic skin bacterial extract is in lysate form. The heterotrophic skin bacterium or heterotrophic skin bacterial extract may be in a lyophilised, freeze-dried or lysate form. Preferably, the heterotrophic skin bacteria is a Micrococcaceae and/or the heterotrophic skin bacterial extract is derived from a Micrococcaceae bacterium. More preferably, the heterotrophic skin bacteria or heterotrophic skin bacterial extract is, or is derived from, a saprotrophic bacterium. Even more preferably, the heterotrophic skin bacterium is a Micrococcus and/or the heterotrophic skin bacterial extract is derived from a Micrococcus bacterium. Most preferably the Micrococcus bacterium may be Micrococcus luteus (M. luteus). As will be appreciated by the skilled person, M. luteus is a Gram-positive saprotrophic coccus bacterium which is part of the normal microbiota of mammalian skin. Preferably the composition comprises M. luteus and/or a bioactive extract of M. luteus in lysate form. The M. luteus may be as deposited under accession number 22110401.

In a related embodiment the composition is formulated for topical application.

In a further related embodiment, the modulation of cytokine expression causes a modulation of one or more of the following cytokines: IL-6, and/or IL-8, PTX-3, VEGF, and/or MIP-3α. The modulation of cytokine expression in a skin cell may be an increase in secretion of one or more of the following cytokines: IL-6, and/or IL-8, and/or PTX-3 and/or a decrease in secretion of one or more of the following cytokines: VEGF, and/or MIP-3α.

In another embodiment the composition is formulated in the form of a cream, gel, spray, ointment or oil. In another related embodiment the composition further comprises one or more pharmaceutically acceptable ingredients or excipients. The composition may suitably be in the form of a liquid, solution (e.g., aqueous, non-aqueous), suspension (e.g., aqueous, non-aqueous), emulsion (e.g., oil-in-water, water-in-oil), elixir, syrup, electuary, mouthwash, cavity wash, drops, granules, powders, ampoule, bolus, suppository, pessary, tincture, gel, paste, ointment, cream, lotion, oil, foam, spray, mist, or aerosol. The composition may suitably be provided as part of a patch, adhesive plaster, bandage, dressing, or the like which is impregnated with one or more active compounds and optionally one or more other pharmaceutically acceptable ingredients, including, for example, penetration, permeation, and absorption enhancers. The composition may also suitably be provided in the form of a depot or reservoir. The composition may also be provided in the form of coatings for medical devices such as implants, prosthetics, surgical instruments, gloves, catheters, valves, pacemakers and the like.

The compositions according to the aspects of the invention may further comprise one or more pharmaceutically or cosmetically acceptable ingredients or excipients. Pharmaceutically acceptable ingredients are well known to those skilled in the art, and include, but are not limited to, pharmaceutically acceptable carriers, adjuvants, excipients, diluents, fillers, buffers, preservatives, carriers, excipients, diluents, fillers, buffers, preservatives, anti-oxidants, lubricants, stabilisers, solubilisers, surfactants (e.g. wetting agents), masking agents, colouring agents, fragrance agents and penetration agents.

In some cases, the carrier may comprise the medium which has been in contact with the bacterium during culturing. The composition of the medium will have changed during the culture, for example by the secretion of material from the bacterium. The compositions may consist or comprise culture medium in which the bacteria have been grown.

Preferably the composition is formulated for topical administration particularly for use or application to, or on, the skin. The composition may be formulated for topical administration in the form of gels, pastes, ointments, creams, sprays, lotions, and oils, as well as patches, adhesive plasters, bandages, dressings, depots, cements, glues, and reservoirs. Preferably the composition may be formulated for topical administration in the form of a cream, gel, spray, ointment or oil.

Ointments are typically prepared from the composition and a paraffinic or a water-miscible ointment base.

Creams are typically prepared from the extract and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions are typically prepared from the heterotrophic skin bacterium and/or a bioactive extract of the heterotrophic skin bacterium and an oily phase, which may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulsion and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono-or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

The composition may be administered alone or in combination with other treatments, either simultaneously or sequentially. Compositions according to the invention may further comprise other active agents, for example anti-microbial agents such as bactericidal and fungicidal agents in order to prevent the composition from spoiling during storage.

In some embodiments, the composition may be provided as a suspension in a pharmaceutically or cosmetically acceptable excipient, diluent or carrier.

The compositions of the present invention may be formulated as medicaments, that is to say formulated as a medicine, or a medical device. The medicament may include other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g. wetting agents), masking agents, colouring agents, flavouring agents, and sweetening agents. The formulation may further comprise other active agents, for example other therapeutic or prophylactic agents.

Media suitable for culturing bacteria (such as M. luteus) is well known to those of skill in the art. As used herein the terms “media” and “medium” encompasses any nutrient containing liquid in which bacteria may be supported, kept alive, grown and/or expanded. The media may contain the minimal nutrients to support bacterial life, and optionally other nutrients. Exemplary nutrients contained within the broth include sugar, magnesium, phosphate, phosphorous and sulphur. The media may be made to, or modified from, a combination of nutrients that is well known in the art, such as tryptic soy agar/broth. Media may be obtained pre-mixed from a commercial source or may be made in-house.

In embodiments, the composition is cell free and does not contain any live bacterial cells. In such embodiments, the whole bacterial cells may have been removed from the media, for example by centrifugation and/or filtration (or other suitable method for removing live bacteria). For example, the bacteria may be removed by sedimenting them from the media in a centrifuge at 15,000×g for a period of time sufficient for substantially all of the bacteria to sediment from the media. The media may be filtered using a microporous filter with pores of a suitable size to remove substantially all of the bacteria from the media. These methods may remove intact bacteria, and may also remove bacterial debris, if the extract is derived by cell lysis.

The composition may be sterile. That is to say that the composition has been subject to a sterilisation process, such as irradiation, heat, chemicals, pressure or filtration, or any combination thereof. For example, the composition may be filter sterilized, a term which will be understood by the skilled person. Filter sterilization makes use of a 0.22 micron filter, as will be understood by the skilled person. Sterilisation procedures must be adapted so as not to damage or reduce the efficacy of the heterotrophic skin bacteria and/or heterotrophic skin bacterial extract. In the case of media containing an extract, the media may have been sterilised before the heterotrophic skin bacteria were introduced and cultured, and also after the bacteria had been removed from that media.

In some cases, the extract of the composition contains substantially no intact bacteria. The composition may also be substantially free from lysed bacteria or bacterial fragments. The intact bacteria and/or lysed bacteria or bacterial fragments may have been separated from the extract. Separation may occur by any suitable means known in the art, such as centrifugation or filtration. By “substantially free from” we mean that the extract contains no or minimal contamination of non-secreted bacterial components, such as whole bacteria, lysed bacteria, or bacterial fragments. Thus, the composition may contain 100% extract, at least 99% extract, at least 95% extract, at least 90% extract, at least 85% extract, at least 80% extract, at least 75% extract or at least 70% extract. The extract may comprise additional components of non-bacterial origin, such as carrier solutions, other active agents, or preservatives, as described herein.

Compositions as described herein may be prepared by culturing the heterotrophic skin bacteria in media, separating the heterotrophic skin bacteria from the media, and preparing a composition from the media. The heterotrophic skin bacteria may be cultured under anaerobic conditions. The heterotrophic skin bacteria may be cultured at a temperature above the normal temperature of the human body. The heterotrophic skin bacteria may be cultured at 30° C., 31° C., 32° C., 33° C., 34° C., 35° C., 36° C., 37° C., 38° C., 39° C., 40° C. or 41° C. Preferably the bacteria are cultured at 37° C. The bacteria may be cultured in the media for 1 day, 2 days, 3 days, 4 days, 5 days, 6 days, 7 days, 8 days, 9 days, 10 days, 11 days, 12 days, 13 days or 14 days. The bacteria or lysed bacteria or fragments may be separated from the media by centrifugation, such as centrifugation at 15000×g. The media may be separated from the heterotrophic skin bacteria, lysed heterotrophic skin bacteria or fragments of heterotrophic skin bacteria by filtration. The media may be separated by a combination of filtration and centrifugation. The media may be subject to sterilisation, before or after the heterotrophic skin bacteria are removed. For example, following separation of the media from the whole bacteria, lysed bacteria or bacterial fragments, the media may be subject to sterilisation. The media may be subject to concentration, such that the proportion of the heterotrophic skin bacterial extract increases relative to the total volume of media. Concentration may occur by any method known in the art, such as evaporation. The heterotrophic skin bacteria extract may be separated from the media. Any method of separating material from a carrier solution may be used. For example, the heterotrophic skin bacterial extract may be separated from the media by chromatography, crystallisation, distillation, drying, electrophoresis or precipitation. Once isolated from the media, or concentrated in the media, the extract may be dissolved or diluted in a carrier, or otherwise formulated into a composition as disclosed herein.

In an additional embodiment of the invention there is provided a composition comprising a heterotrophic skin bacterium and/or heterotrophic skin bacterial extract for use in the prevention and/or treatment of skin damage caused by UV light.

In a further embodiment of the invention there is provided the use of a composition comprising a heterotrophic skin bacterium and/or heterotrophic skin bacterial extract for use in the manufacture of a medicament for the prevention, management or treatment of skin damage caused by UV light.

In a yet further embodiment of the invention there is provided a composition comprising a heterotrophic skin bacterium and/or heterotrophic skin bacterial extract for use in preventing, managing or treating a skin condition caused by exposure to UV light. In a related embodiment the skin condition is PLE.

In embodiments, the composition may be for the prevention of skin damage caused by UV light or a skin condition caused by exposure to UV light. In such embodiments, the composition may be applied or administered before/prior to exposure to UV light. As will be appreciated by the skilled person by before/prior to exposure to UV light, it is meant an amount of UV light sufficient to cause skin damage or a skin condition, for example PLE.

In a further related embodiment, the composition is formulated to be applied topically to the skin. In another related embodiment the skin condition is a VEGF and/or MIP-3α mediated skin condition. In a further related embodiment, the skin condition is a IL-6, and/or IL-8, and/or PTX-3 mediated skin condition.

In another embodiment of the invention, there is provided a composition comprising a heterotrophic skin bacterium and/or heterotrophic skin bacterial extract for use in a cosmetic preparation for application to skin damaged by UV light. Typically, such cosmetic preparations are marketed as ‘after sun’ and are for application to the skin after prolonged exposure to the sun.

Compositions and formulations according to the invention may further comprise other active agents, for example other anti-bacterial agents such as bactericidal agents.

In some embodiments the composition may comprise at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 1 0.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% by weight of the heterotrophic skin bacteria and/or heterotrophic skin bacterial extract.

In some embodiments the composition may comprise, one of at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to 10 about 5%, by weight of the heterotrophic skin bacteria and/or heterotrophic skin bacterial extract.

In another embodiment the invention provides a method of preventing, managing or treating a skin condition caused by exposure to UV light, the method comprising applying a composition as defined in any other embodiment to the skin. The skin condition may be PLE. Also provided is a method of preventing and/or treating skin damage caused by UV light, the method comprising applying a composition as defined in any other embodiment to the skin. In embodiments, the composition may be for the prevention of skin damage caused by UV light or a skin condition caused by exposure to UV light. In such embodiments, the composition may be applied or administered before/prior to exposure to UV light. As will be appreciated by the skilled person by before/prior to exposure to UV light, it is meant an amount of UV light sufficient to cause skin damage or a skin condition, for example PLE.

In a further embodiment the invention provides a method of modulating cytokine expression in a skin cell, the method comprising applying a composition as defined in any other embodiment.

The heterotrophic skin bacterial preparations according to the invention may be formulated as pharmaceutical compositions for clinical use and may comprise a pharmaceutically acceptable carrier, diluent or adjuvant. They may be formulated for topical administration.

Administration is preferably in a prophylactically or therapeutically effective amount, this being an amount sufficient to show benefit to the individual. The actual amount administered, and rate and time-course of administration will depend on the nature and severity of the disease being treated. Prescription of treatment, e.g. decisions on dosage etc., is within the responsibility of general practitioners and other medical doctors, and typically takes account of the disorder to be treated or prevented, the condition of the individual patient, the site of delivery, the method of administration and other factors known to practitioners. Examples of the techniques and protocols mentioned above can be found in Remington's Pharmaceutical Sciences, 20^{th} Edition, 2000, pub. Lippincott, Williams & Wilkins. It will be appreciated by the skilled person that appropriate dosages of the active compounds and compositions comprising the active compounds can vary from patient to patient.

The compositions of the present invention may be formulated as medicaments, that is to say formulated as a medicine or a medical device. The medicament may include other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g. wetting agents), masking agents, colouring agents, flavouring agents, and sweetening agents. The formulation may further comprise other active agents, for example other therapeutic or prophylactic agents.

The compositions of the present invention may be formulated as cosmetics, that is to say formulated as a cosmetics product. The cosmetics product may include other cosmetically acceptable ingredients well known to those skilled in the art, including, but not limited to, cosmetically acceptable carriers, excipients, diluents, fillers, buffers, preservatives, anti-oxidants, lubricants, stabilisers, solubilisers, surfactants (e.g. wetting agents), masking agents, colouring agents, fragrance agents.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1 is a graph showing the cytotoxic effects of UVB are dose dependent. NHEKs (p3-5) cultured to 80% confluency were irradiated at 5-30 mJ/cm². 24 h post-irradiation cell viability (n=3) was assessed via trypan blue. Data are presented as mean±SD. **P<0.01, *P<0.05, as determined via one-way ANOVA followed by Tukey test.

FIG. 2 is two graphs showing that UVB has no significant effect on IL-10/IL-12 secretion. Supernatants collected 24 h post-irradiation (5-30 mJ/cm², p3-5) were assayed for (A) IL-10 (n=3) and (B) IL-12 (n=2). Data presented as mean±SD. Significance assessed via one-way ANOVA followed by Dunnett's test.

FIG. 3 is a collection of array blot images, and graphs, showing that UVB exerts dose dependent effects on inflammatory mediators. FIG. 3A are images of array blots post assaying for cytokine release from pooled supernatants (n=3, p3-5) collected 24 h post-irradiation with 0 (i), 10 (ii), 20 (iii) and 30 mJ/cm² (iv). FIG. 3B is a graph showing changes in protein secretion relative to non-irradiated control. FIG. 3C are graphs showing the quantification of dose dependent changes in pro-inflammatory mediators (I, ii and iii); IL-6 (i), IL-8 (ii) and MIP-3α (iii), proangiogenic markers (iv and v); VEGF (iv), MMP-9 (v) and acute phase protein; PTX3 (vi) via imageJ.

FIG. 4 is a collection of graphs showing the validation of cytokine array analyses via ELISA. Supernatants collected 24 h post-irradiation (5-30 mJ/cm², n=3, p3-5) were assayed for (A) IL-6, (B) IL-8, (C) MIP-3α (D) and PTX-3 (E). Data are presented as mean±SD. ****P<0.0001, **P<0.01, *P<0.05, as determined via one-way ANOVA followed by Dunnett's test.

FIG. 5 is a graph showing that the protective effects of Micrococcus luteus are dose dependent. NHEKs (p3-5) were cultured to 80% confluency, treated with media±M. luteus 10⁴/10² CFU/ml for 90 mins and irradiated at 5-30 mJ/cm². 24 h post-irradiation, cell viability was assessed by trypan blue. Data are presented as mean±SD. ****P<0.0001, *P<0.05, as determined via two-way ANOVA followed by Tukey test.

FIG. 6 is two graphs that show UVB promotes M. luteus adherence to keratinocytes. NHEKs (p3-5) were cultured to 80% confluency, treated with media±M. luteus 10⁴/10² CFU/ml for 90 mins and irradiated at 5-30 mJ/cm². Adherence of M. luteus at 1×10⁴ CFU/ml (A, n=3) and 1×10² CFU/ml (n=4, B) was assessed 24 h post-irradiation. Data are presented as mean±SD. *P<0.05, as determined via one-way ANOVA followed by Dunnett's test.

FIG. 7 is three graphs showing that M. luteus exacerbates UVB induced pro-inflammatory cytokine production. Supernatants collected 24 h post-irradiation±M. luteus application (5-30 mJ/cm², n=3, p3-5) were assayed for (A) IL-6, (B) IL-8 and (C) PTX-3. Data are presented as mean±SD. ****P<0.0001, ***P<0.001,**P<0.01 and *P<0.05, as determined via two-way ANOVA followed by Tukey test.

FIG. 8 is two graphs that show M. luteus modulates UVB induced VEGF secretion. Supernatants collected 24 h post-irradiation±M. luteus application (5-30 mJ/cm², n=3, p3-5) were assayed for (A) VEGF and (B) MIP-3α. Data are presented as mean±SD. **P<0.01, *P<0.05, as determined via two-way ANOVA followed by Tukey test.

FIG. 9 is a graph showing that Micrococcus luteus exerts protective effects on UVR treated differentiated keratinocytes. Differentiated NHEKs (p2-3) treated with media±M. luteus 10⁴/10² CFU/mL for 1 h and irradiated at 5-30 mJ/cm². 24 h post-irradiation, cell viability was assessed by trypan blue. Data are presented as mean±SD. ****P<0.0001, ***P<0.001, **P<0.01, *P<0.05, as determined via two-way ANOVA followed by Tukey test.

FIG. 10 is two graphs that show the adherence of M. luteus to keratinocytes in response to UVB exposure. NHEKs (p2-3) treated with media±M. luteus 10⁴/10² CFU/mL for 1 h and irradiated at 5-30 mJ/cm². Adherence of M. luteus at 1×10⁴ CFU/ml (A, n=3) and 1×10² CFU/ml (n=3, B) was assessed 24 h post-irradiation. Data are presented as mean±SD. Significance was assessed via one-way ANOVA followed by Dunnett's test.

### EXAMPLES

### Example 1: Evaluation of the Cytotoxic Effects of UVB on Epidermal Keratinocytes

### Materials and Methods

### Keratinocyte Culture

Pooled normal human epidermal keratinocytes (NHEK, n=3, juvenile foreskin, Promocell C-12005, passage 1-5) were routinely cultured in keratinocyte growth medium (KGM-2, Promocell). Cells were seeded into 12-well plates (2-5×10⁴ cells/well) and incubated (37° C., 5% CO₂) until 80% confluency was reached. Cells were washed with phosphate buffered saline (PBS, Gibco) and either resuspended in PBS for UV irradiation or inoculated with bacteria as described below.

### UV Irradiation

A broadband UVB emitting TL-12 lamp (Philips) was employed emitting a maximum power output of 20W and wavelengths from 270 - 400 nm (peak: 313 nm). Using a UVX radiometer (UV products, California) coupled to a UVX-31 detector, irradiance output was assessed for at least five minutes prior to irradiation until levels stabilised to 0.55±0.01 mW/cm². Irradiation time was then calculated as radiant exposure (mJ/cm²)/(irradiance×calibration factor), in which the calibration factor was 0.63 and radiant exposure doses from 5-30 mJ/cm² were applied for experimentation. Cell monolayers resuspended in PBS were placed at a distance of 16 cm from the light source, with half the plate covered in foil to act as a non-irradiated control. Cells were exposed at doses described above, media was replaced with KGM-2 and incubated for 24 h (37° C., 5% CO₂).

### Cell Viability and Bacterial Adherence

24 h post irradiation, cells were washed 3×with PBS and trypsin (0.04%) Ethylenediaminetetraacetic acid (EDTA, 0.03%, Promocell) was applied for 7 minutes (37° C., 5% CO₂). An equal volume of trypsin neutralising solution (TNS) was then applied to cells and a sample was mixed with an equal volume of trypan blue (Gibco). Cell viability was then recorded using a haemocytometer (improved Neubauer). Alternatively, cell suspensions were diluted to 1 mL in PBS, serially diluted in TSB to 10⁻⁵ and aliquots at 20 μL were applied in triplicate to TSB agar to ascertain microbial adherence to NHEKs.

### UV Modulated Inflammatory Cytokine Concentrations

The relative expression of 105 inflammatory cytokines was assessed using the Human XL Cytokine Array Kit (R&D Systems, Minneapolis, MN, USA), following manufacturer's instructions, in pooled supernatant samples (n=3, NHEK p3-5) treated with 10-30 mJ/cm² doses of UVB.

Supernatants were assayed for human total interleukin-6 (IL-6), IL-8, IL-10, IL-12, pentraxin-3 (PTX-3), macrophage inflammatory protein-3α (MIP-3α), and vascular endothelial growth factor (VEGF) by ELISA according to the manufacturer's protocol (Duoset, R and D systems, Biotechne).

### Statistical Analyses

Data were processed utilising Excel software (Microsoft) and analyses were performed using Prism (GraphPad Software, California, US). All experiments were performed at least in triplicate, unless otherwise stated, and data were analysed using Student's t-test (when comparing two groups) or general linear model (GLM) followed by one-way or two-way ANOVA and Tukey/Dunnett's test (when comparing three or more groups).

### Results

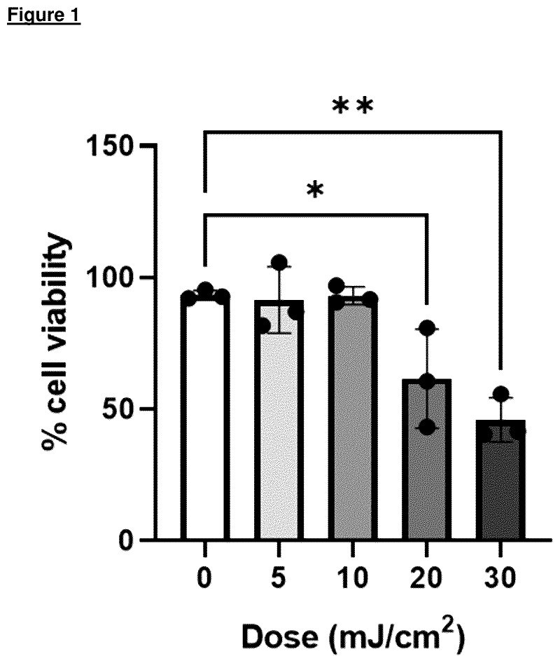

Previous studies have provided evidence of the dose dependent cytotoxic effects of UVB on epidermal keratinocytes. Our results corroborate with these findings, indicating doses of UVB>20 mJ/cm² were sufficient to induce a 34% reduction in cell viability relative to the non-irradiated control as show in FIG. 1. These cytotoxic effects are thought to be attributed to an imbalance between immunosuppressive (IL-10) and pro-inflammatory (IL-12) cytokines.

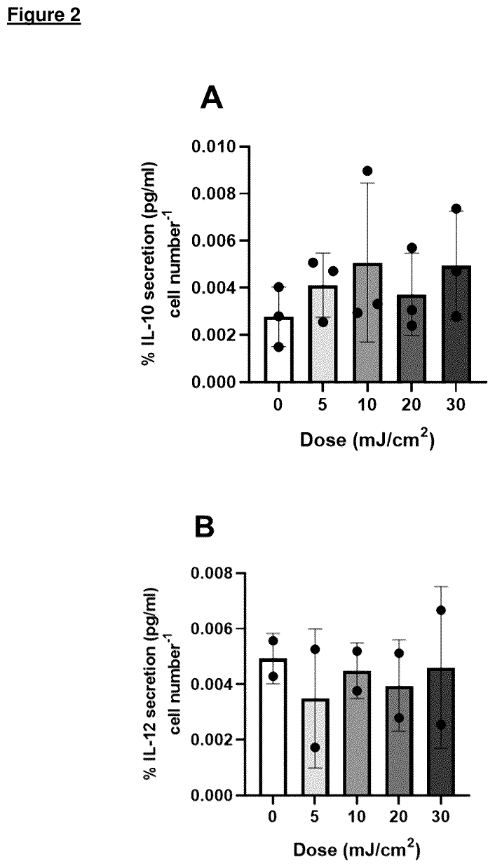

However, FIG. 2 shows that UVB exerted no significant effect on IL-10 or IL-12 secretion (FIG. 2). Therefore, UVB dependent cytokine secretion was characterised using a cytokine array.

### Example 2: Assessment of Dose Dependent Changes in Cytokine Secretion

### Materials and Method

The methods were carried out as above for Example 1.

### Results

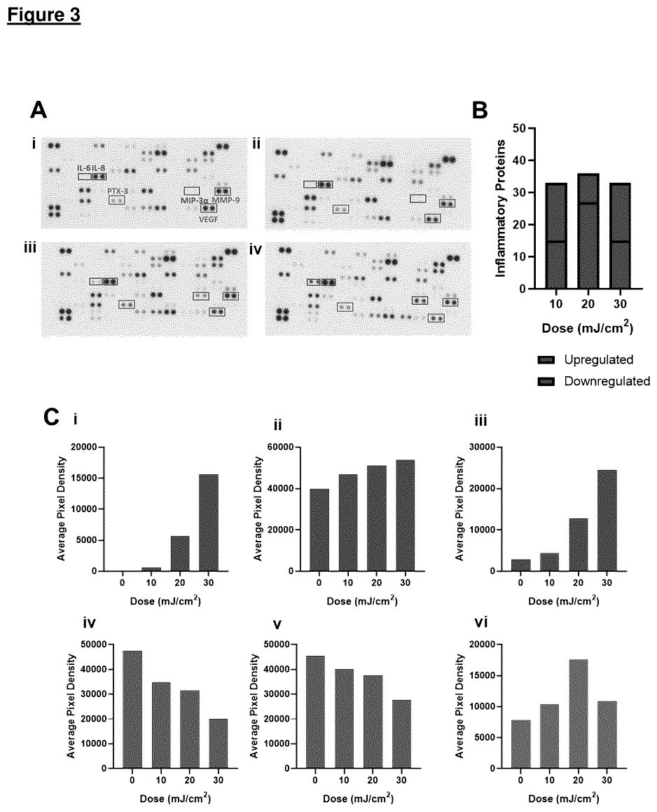

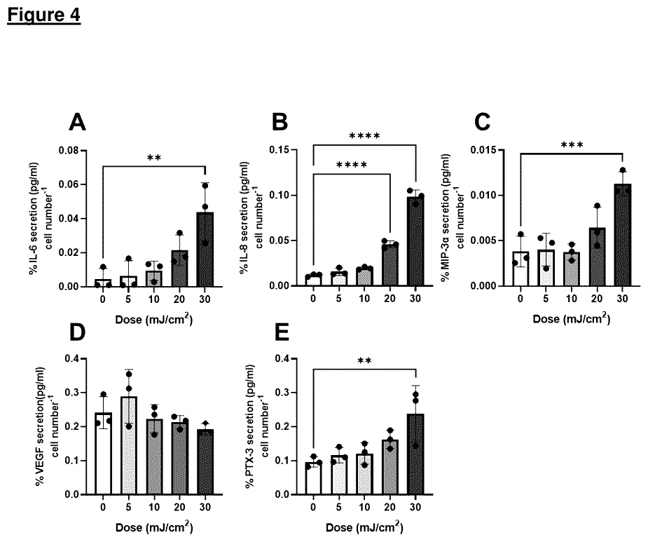

A cytokine array was employed to examine the dose dependent effects of UVB on the secretion of 107 cytokines. Assessment of changes in relative pixel density revealed basal expression of 45 inflammatory proteins. FIG. 3B shows that irradiation at 10 mJ/cm², 20 mJ/cm² or 30 mJ/cm² resulted in the increased secretion of 15, 27 and 15 proteins respectively relative to the non-irradiated control. Quantification of relative pixel density of blots as shown in FIG. 3A revealed a dose dependent change in a range of inflammatory mediators which is shown in FIG. 3C and these changes were verified quantitatively via ELISA as included in FIG. 4. Dose dependent increases in pro-inflammatory mediators including IL-6, IL-8 and MIP-3α were observed as evidenced in FIG. 4A-C. Concomitantly, dose dependent increases in the secretion of the innate immune sensor and regulator, PTX-3 were also observed and shown in FIG. 4E. Comparatively, the dose dependent decreases in VEGF secretion included in FIG. 3C, iv were not validated via ELISA shown in FIG. 4D as this trend was attributed to UVB induced cell cytotoxicity. Skin microbiome mediated effects on UVB dependent cytotoxicity and inflammation were then characterised.

### Example 3: M. luteus Exerts a Protective Effect Against Cytotoxic Doses of UVB

### Materials and Method

### Bacterial Co-Culture

Micrococcus luteus was routinely cultured in tryptic soy broth (TSB, Oxoid) for 48 h at 37° C., cells were washed twice with PBS and resuspended to 1×10⁴−1×10⁶ CFU/mL in KGM. To confirm viable counts, inocula were serially diluted and applied at 20 μL in triplicate to TSB/WCB agar. Media±M. luteus was then applied for 90 minutes (37° C., 5% CO₂) and non-adherent cells washed with PBS twice. Cell monolayers were then UV irradiated as described below.

All other methods were carried out as above in Example 1.

### Results

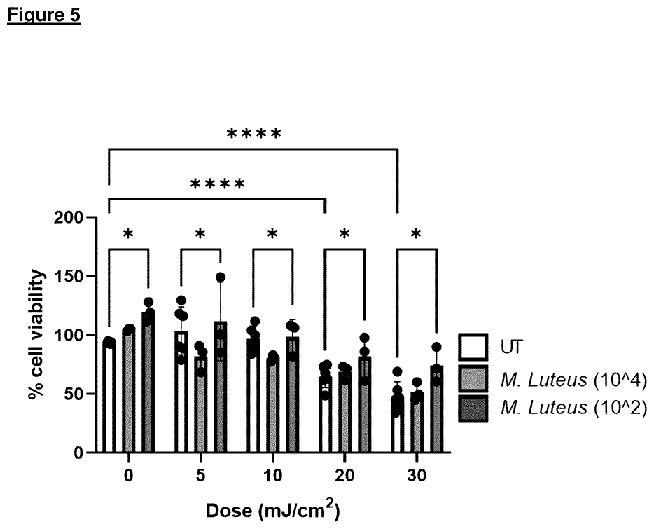

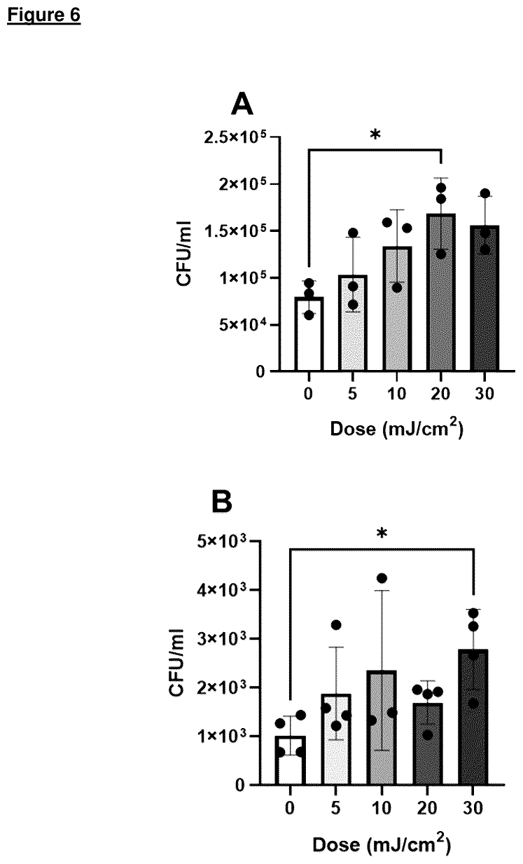

Previous studies document that M. luteus is capable of repairing UV induced DNA damage. However, whether it confers this protection to skin remains to be characterised. M. luteus induced photoprotection was bacterial load dependent. FIG. 5 demonstrates that inoculation at 1×10² CFU/mL resulted in a 53% increase in cell viability relative to the 30 mJ/cm² non-bacterially treated control. Comparatively inoculation at 1×10⁴ CFU/mL exerted no significant effect on cell viability. Interestingly, this photoprotective effect was also accompanied by elevated microbial adherence to NHEKs. Application of UVB at 30 mJ/cm¹ to NHEKs inoculated with 1×10⁴ or 1×10² CFU/ml M. luteus resulted in 97% and 174% increases in adherence relative to non-irradiated controls respectively as shown in FIG. 6. Without wishing to be bound be theory, the inventors believed that this elevated adherence may modulate UVB associated inflammation, thus conferring photoprotection. Therefore, the effects of M. luteus on UVB modulated inflammatory proteins were assessed.

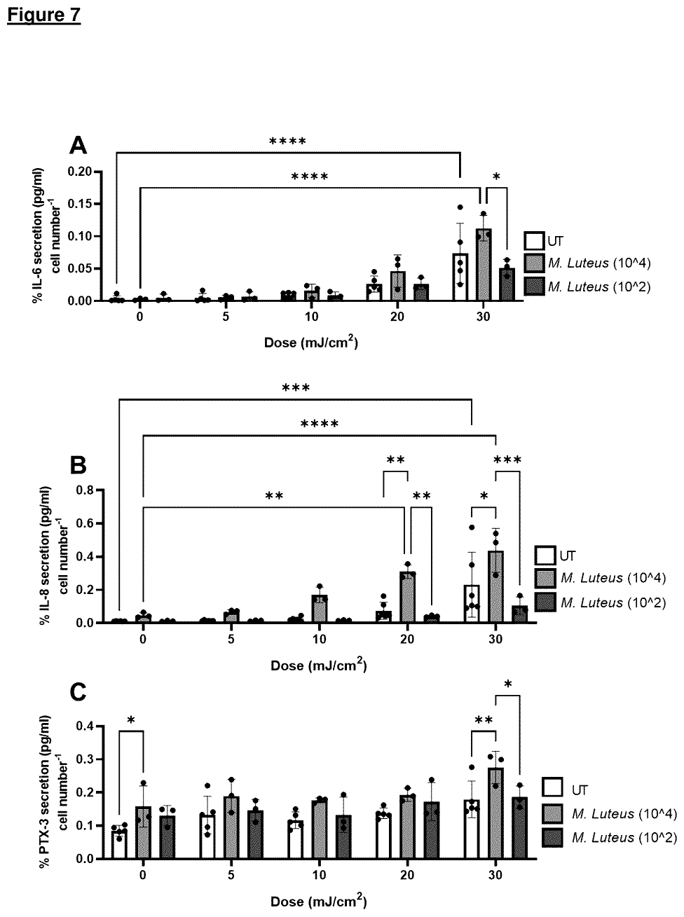

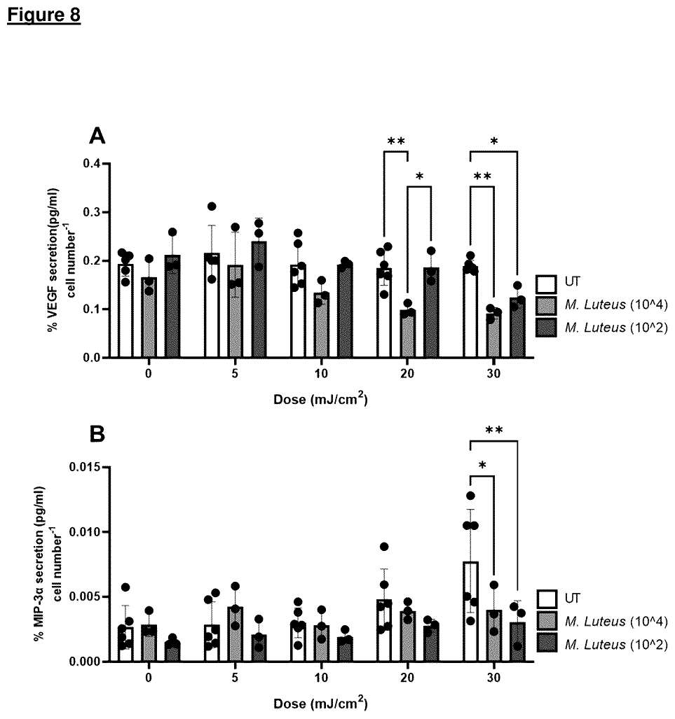

The inflammatory response to M. luteus is thought to be toll like receptor-4 dependent, resulting in the elevated production of cytokines including IL-8. The inventors observed this response was M. luteus dose dependent. FIG. 7B demonstrates that high doses of M. luteus (1×10⁴ CFU/mL) resulted in elevated secretion of IL-8, a response exacerbated by cytotoxic doses of UVB (30 mJ/cm²). This trend was mirrored with IL-6 and PTX-3, where cytokine secretion was significantly elevated in cultures treated with 30 mJ/cm² UVB+1×10⁴ CFU/mL M. luteus compared to those treated with 30 mJ/cm²±1×10² CFU/mL M. luteus as shown in FIG. 7. Comparatively, inoculation at either microbial density resulted in significant reductions in UVB (30 mJ/cm²) mediated secretion of VEGF (shown in FIG. 8a) and MIP-3α (shown in FIG. 8b). Indicating, these factors could pay important roles in M. luteus associated photoprotection.

### Example 4: M. luteus has Photoprotective Effects on Differentiated Keratinocytes

### Materials and Method

Materials and methods were carried out as above in Examples 1 and 3.

### Results

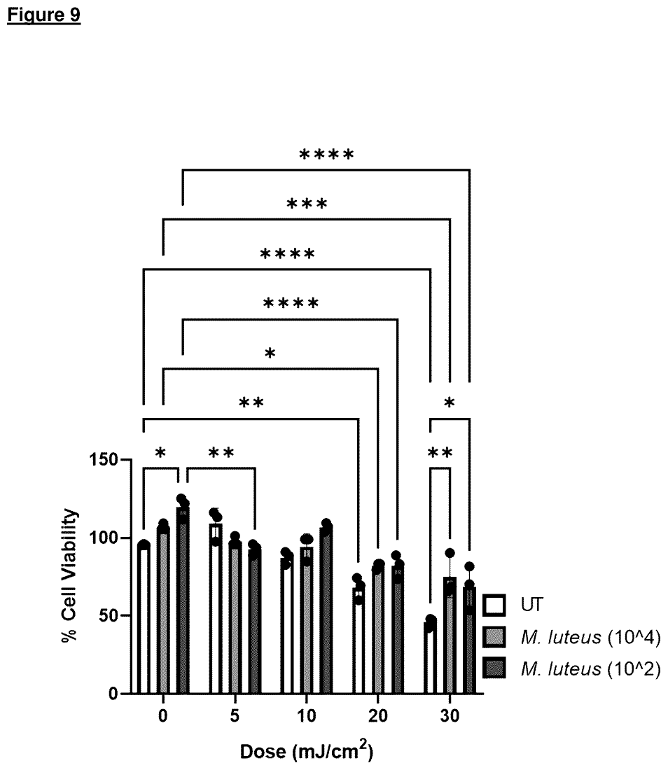

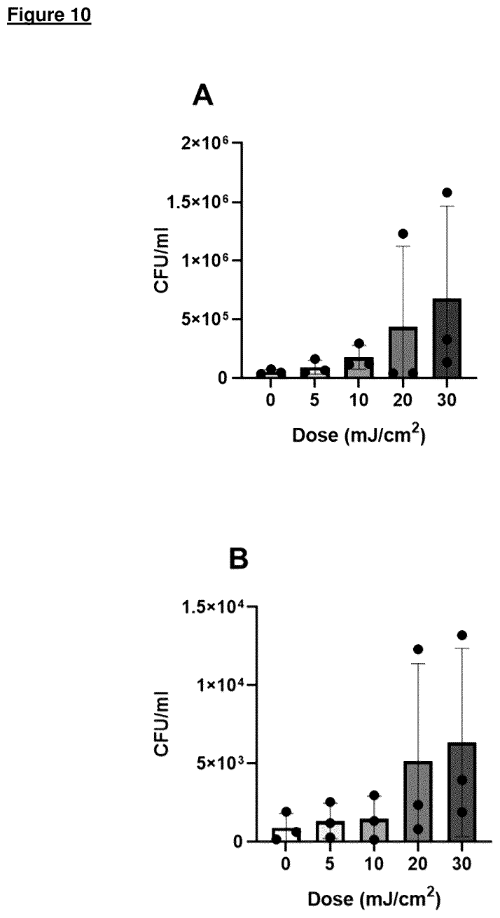

The epidermis is formed of multiple layers of keratinocytes at varying stages of differentiation. Actively proliferating keratinocytes form the basal layer, whilst differentiated keratinocytes act at the surface of skin, orchestrating initial responses to both UVR and pathogens. To ensure the model utilised in this study was more physiologically relevant, the protective effects of M. luteus on UVR treated differentiated keratinocytes was assessed. As with undifferentiated keratinocytes, FIG. 9 demonstrates that UVR exerted a dose dependent reduction on cell viability, resulting in a 52% reduction following irradiation at 30 mJ/cm² relative to the untreated control. M. luteus effectively mitigated the cytotoxic effects of UVR (30 mJ/cm²), resulting in a 63% and 49% increase in viability following treatment with high (1×10⁴ CFU/mL) and low (1×10² CFU/mL) dose M. luteus respectively relative to the 30 mJ/cm² non-bacterially stimulated control. Additionally, FIG. 10 shows that this response was accompanied by a trend towards a UVR dose dependent increase in M. luteus adherence to keratinocytes.

### Overall Conclusions

Data regarding the immune systems response to UVR has been published previously. These data have mostly been generated in humans and the specific cell type producing the immune mediators has not been characterised. The present work specifically investigates the keratinocyte response to UVR. Keratinocytes were chosen because they are a major component of the innate immune system in skin which has to date been poorly characterised in terms of its response to UVR. Secondly, keratinocytes are likely the main cell type that interacts with the microbiome (since the microbiome is thought to reside mostly on the surface of the skin).

To date, it has been shown that the array of cytokines produced by keratinocytes is vast. Furthermore, data included in this document shows that the cytokine response can be modified by at least one bacterium, M. luteus. This can be used to shift the balance between Th1 and Th2 cytokines demonstrating the usefulness of the therapeutic in conditions such as PLE, which currently affects ˜18% of Europeans.

It was surprisingly found that heterotrophic skin bacteria were able to modulate the skin cell response to UV exposure. The inventors found that inoculating skin cells with M. luteus caused an increased in survival of skin cells after exposure to UV light. This was surprisingly found to be a consequence of the ability of M. luteus to modulate the immune response of a skin cell by reducing the expression of certain cytokines and increasing the expression of a different set of cytokines.

The forgoing embodiments are not intended to limit the scope of the protection afforded by the claims, but rather to describe examples of how the invention may be put into practice.

### Biological Deposits

The application refers to the following indications of deposited biological material:
Name: National Collection of Type Cultures
Address: UK Health Security Agency, Porton Down, Salisbury, SP4 0JG United Kingdom
Accession Date: 4 Nov. 2022
Accession Number: 22110401
Species: Micrococcus luteus
Depositor: The University of Manchester, Oxford Road, Manchester, M13 9PL United Kingdom

1. A composition comprising a heterotrophic skin bacterium and/or a heterotrophic skin bacterial extract for use in the modulation of cytokine expression in a skin cell.

2. The composition according to claim 1, wherein the heterotrophic skin bacterial extract is in lysate form.

3. The composition according to claim 1, wherein the heterotrophic skin bacterium is derived from a Micrococcus bacterium.

4. The composition according to claim 3, wherein the Micrococcus bacterium is Micrococcus luteus.

5. The composition according to claim 1, wherein the composition is formulated for topical application.

6. The composition according to claim 1, wherein the modulation of cytokine expression causes modulation in secretion of one or more of the following cytokines: IL-6, and/or IL-8, PTX-3, VEGF, and/or MIP-3α, in the skin cell

7. The composition according to claim 1, wherein the modulation of cytokine expression causes an increase in secretion of one or more of the following cytokines: IL-6, and/or IL-8, and/or PTX-3, in the skin cell.

8. The composition according to claim 1, wherein the modulation of cytokine expression causes a decrease in secretion of one or more of the following cytokines: VEGF, and/or MIP-3α, in the skin cell.

9. The composition according to claim 1, wherein the composition further comprises one or more pharmaceutically acceptable ingredients or excipients.

10. The composition according to claim 9, wherein the composition is formulated in the form of a cream, gel, spray, ointment or oil.

11. The composition according to claim 1, for use in the prevention and/or treatment of skin damage caused by UV light.

12. The composition according to claim 1 for use in a cosmetic preparation for application to skin damaged by UV light.

13. The composition according to claim 1, for use in preventing, managing or treating a skin condition caused by exposure to UV light.

14. The composition according to claim 13 wherein the skin condition is PLE.

15. The composition according to claim 13, wherein the composition is formulated to be applied topically to the skin.

16. The composition according to claim 11, wherein the composition is administered prior to exposure to UV light.

17. The composition according to claim 13, wherein the skin condition is a VEGF and/or MIP-3α mediated skin condition.

18. The composition according to claim 13, wherein the skin condition is a IL-6, and/or IL-8, and/or PTX-3 mediated skin condition.

19. The composition according to claim 1, wherein the heterotrophic skin bacterium and/or heterotrophic skin bacterial extract comprises or is derived from Micrococcus luteus (22110401).
